(19) United States
(12) Patent Application Publication
(10) Pub. No.: US 2009/0163808 A1
(43) Pub. Date: Jun. 25, 2009

(54) Echographic imaging device, and apparatus for detecting and destroying solid concretions, which apparatus incorporates such a device

(76) Inventors: Andre Peyrard, Saint-Christo-En Jarez (FR); Rene Chatre, Venissieux (FR); Olivier Nallet, Lyon (FR)

Correspondence Address: CLARK & BRODY, 1090 VERMONT AVENUE, NW, SUITE 250, WASHINGTON, DC, 20005, US

(21) Appl. No.: 12/314,535

(22) Filed: Dec. 12, 2008

(30) Foreign Application Priority Data
Dec. 14, 2007 (FR) 07 59852

(51) Int. Cl. A61B 17/225; A61B 8/00

(52) U.S. Cl. 600/439; 601/4

(57) ABSTRACT

The present invention relates to a real-time echographic imaging device comprising at least one echographic probe incorporating an ultrasound transducer connected electrically to a current generator for generating ultrasound waves. Said echographic imaging device further comprises an arm of longitudinal axis X-X′ and supporting the echographic probe, which arm is mounted to move in translation along its longitudinal axis X-X′ and in rotation thereabout via motor-driven displacement means. Said device further comprises control means for controlling the motor-driven displacement means, and force-monitoring means for measuring the application pressure with which the probe as moved by the moving arm is applied against a surface, in particular against a patient's body, and servo-control means for servo-controlling the displacement means for moving the arm so as to maintain said application pressure constant independently of the movements of the surface and of the movements of the probe relative to said surface.

### FIELD OF THE INVENTION

The present invention relates to an echographic imaging device comprising at least one echographic probe with application pressure that is controlled. It also relates to apparatus for detecting and destroying solid intracorporeal concretions, which apparatus incorporates such an echographic imaging device, in particular for lithotripsy treatment.

### BACKGROUND OF THE INVENTION

Echographic imaging devices have long been known that comprise a probe formed essentially of an applicator incorporating an ultrasound transducer connected to a current generator so as to emit ultrasound, and receiver means for receiving the reflected ultrasound waves, making it possible, after filtering and electronic processing, to obtain images of a volume through which the ultrasound waves have passed and in which they have been reflected. Such echographic imaging technologies are used, in particular, in various medical uses for observing organs and tissue or indeed foreign bodies inside a patient's body.

Such uses include, in particular, lithotripsy, which consists essentially in detecting calculi such as “kidney stones”, “gallstones”, or “salivary stones”, and in destroying them by acoustic shock waves generated by a generator. In that use, echographic imaging devices are employed, generally in combination with X-ray imaging means, for detecting the presence of calculi in a patient's body and also the positions of such calculi, and then, during the treatment, for tracking changes in state of such calculi and any movement thereof.

The same devices can also be used for orthopedic uses consisting in delivering shock waves to bone or cartilage structures with a view to treating pathologies of the arthrosis or tendonitis types, for example. Other uses are being investigated, and the present invention is not limited merely to extracorporeal lithotripsy.

In any event, the therapeutic principle requires:
the anatomical target, i.e. the calculus, to be put into correspondence with the active focus of the shock wave generator;
the target to be tracked, if possible continuously (in “real time”), in order to be able to detect any loss of correspondence resulting from the target moving during the treatment (untimely movement of the patient, natural breathing movement, etc.).

Both of the above-mentioned imaging technologies are commonly used jointly because they are complementary for the function of putting the target into correspondence with the focus of the generator, due to the fact that certain calculi can be either X-ray transparent or echo-transparent, and must therefore be located by the technology that enables them to be viewed.

In addition, for the tracking function, which ideally requires the calculus to be viewed continuously, echographic technology is the natural choice because of its innocuousness, in particular due to the absence of ionizing radiation. In any event, the therapeutic principle requires the quality of the images to be good.

The quality of the echographic images obtained depends to a large extent on the acoustic coupling between the probe and the patient. At the frequencies used in the medical field (megahertz (MHz)), ultrasound waves do not pass through the air, and echographic examinations long used to be and still are in most cases performed by a physician who supports the echographic probe manually and applies it to a patient's body.

In order to improve the coupling or in order to optimize the viewing angles and the quality of the images, it is very frequent for physicians to use a water-based gel that makes it possible to guarantee good transmission of the acoustic waves on contact with the patient. It is also very frequent for them also to vary the contact pressure of the probe on the patient's skin. In certain circumstances, e.g. for deep exploration or exploration of organs that are difficult to access, said pressure can be sufficiently high to be uncomfortable for the patient.

The application pressure with which the echographic probe is applied must also be adapted as a function of the patient being examined. For example, a child or a thin patient does not have the same tolerance to the contact force of the echographic probe as an adult or as a corpulent patient.

Finally, manual application of the echographic examination is handicapping and tiring for the physicians, in particular for examinations and treatments that are lengthy, as lithotripsy treatments can be.

Patent WO 90/01904 describes a mechanical system that makes it possible to track the movements of a target, in particular a renal calculus, by using echographic imaging means in lithotripsy apparatus.

In the system described in Document WO 90/01904, the echographic probe support is implemented by a mechanical device without operator control. That mechanical device makes it possible to position and to apply the echographic probe against the body of a patient in order to perform an echographic scan for identifying a calculus. Since the shock wave generator of the lithotripsy apparatus described in that document has a three-dimensionally fixed horizontal position, the efforts exerted by the forces of gravity on the echographic probe and therefore the forces exerted on a patient are therefore constant.

However, if the patient moves slightly or if the probe is moved, the quality of the echographic images obtained is particularly affected. Similarly, depending on whether the patient is thin or obese, the contact pressure with which the probe presses against the patient varies and the quality of the echographic images is also affected.

Unfortunately, the mechanical device for supporting the acoustic probe that is described in WO 90/01904 does not make it possible, under such circumstances, to re-establish coupling (and therefore satisfactory images) rapidly and correctly, and is, in certain circumstances, less effective or less practical than manually manipulating the echographic probe, in particular for tracking the movement of a calculus in the body.

In addition, in lithotripsy apparatus of recent generations, the application pressure (contact force) with which the echographic probe is applied can vary during the same examination, due to the shock wave generator being moveable in rotation since it is mounted on a moving support. Then, depending on the angular position of the generator, the forces of gravity on the echographic probe vary and thus the application pressure with which the probe is applied against the patient also varies positively or negatively, and the resulting non-constant pressure has an adverse impact on the quality of the echographic images obtained.

A major technical problem thus remains to be solved consisting in effectively automating the action of moving an echographic probe and of applying it against a patient's body so as to obtain the best possible coupling and the best possible echographic image, regardless of the morphology of the patient and of the position of the probe relative to the patient.

The above-mentioned technical problem includes, in particular, the difficulty of automatically maintaining a constant positive pressure for the pressure with which the echographic probe presses against the patient's body, so as to guarantee the coupling of the probe, while also enabling the practitioner to choose the value for the application pressure with which the probe is applied against the patient and to cause said value to vary as a function of the patient and of the positions of the echographic probe.

Those problems are considerable, in particular in the field of lithotripsy treatment due to the need to make sure that the positions of the calculi under treatment are kept continuously in correspondence with the focus of the shock wave generator, regardless of the position of said generator.

### OBJECT AND SUMMARY OF THE INVENTION

An object of the present invention is therefore to procure a solution to the above-mentioned technical problem.

Another object of the invention is to procure a solution to the above-mentioned technical problem that is simple and effective to implement for the operators/physicians.

Another object of the invention is to procure a solution to the above-mentioned technical problem that is easy to use in the various medical and veterinary uses of echographic imaging.

These objects are achieved in accordance with the present invention by providing an echographic imaging device comprising at least one echographic probe incorporating an ultrasound transducer connected electrically to a current generator for generating ultrasound waves. Said echographic imaging device further comprises a straight arm of longitudinal axis X-X′ and supporting the echographic probe, which arm is mounted to move in translation along its longitudinal axis X-X′ and in rotation thereabout via motor-driven displacement means.

Said device also further comprises electrical control means for electrically controlling the motor-driven displacement means for moving the moving arm, and force-monitoring means for measuring the application pressure with which the probe as moved by the moving arm is applied against a surface, in particular against a patient's body.

The echographic imaging device of the invention finally further comprises servo-control means for servo-controlling the displacement means for moving the arm so as to maintain said application pressure constant independently of the movements of the surface and of the movements of the probe relative to said surface.

The echographic imaging device of the invention thus makes it possible simply and with good performance to apply an echographic probe against a surface, in particular of the body of a patient, in automated manner and at constant pressure. By means of the force-monitoring means, the device continuously computes the application pressure of the echographic probe. The force-monitoring means are advantageously connected to the control means for controlling the displacement means for moving the moving arm of the device that carries the probe, or indeed they are connected directly to said displacement means so as to adjust operation thereof, via the servo-control means, in such a manner as to keep the application pressure of the probe constant.

It should be noted that it is possible to configure the imaging device of the invention in a manner such that regulation of the application pressure of the echographic probe by the moving arm is dependent or not dependent on the three-dimensional position of the probe, and thus on the forces of gravity.

It is possible to implement the motor-driven displacement means for moving the moving arm so as to be servo-controlled relative to a setpoint application pressure value, in which case only the pressure value given by the force-monitoring means governs the servo-control, which takes place independently of the position of the echographic probe.

It is possible, advantageously, to implement the motor-driven displacement means for moving the moving arm so as to be servo-controlled relative to a setpoint voltage or current value for powering said motor-driven displacement means. Said voltage/current value is chosen to correspond to a desired application pressure for applying the probe, in which case the three-dimensional position of the echographic probe and the forces of gravity are involved and are taken into account automatically in regulating the application pressure with which the echographic probe is applied by its moving arm.

Another object of the present invention also consists in procuring apparatus for detecting and destroying solid intracorporeal concretions such as renal, gall bladder, or salivary calculi that includes an echographic imaging device of the invention as defined above. Such apparatus can also be used in the field of orthopedics, in which the shock waves are then used at lower power for treating joint pain.

In its simplest embodiment, said apparatus then comprises a fixed structure supporting imaging means for imaging intracorporeal concretions located in the body of a person or of an animal, which imaging means include at least the echographic imaging device of the invention, and an acoustic shock wave generator configured to reach and destroy said intracorporeal concretions located in the body of a person or of an animal on the basis of location of said intracorporeal concretions by the imaging means.

### BRIEF DESCRIPTION OF THE DRAWINGS

Various other characteristics appear from the following description given with reference to the accompanying drawings which show embodiments of the invention by way of non-limiting example, and in which:

FIG. 1 is a perspective view of a preferred embodiment of an echographic device of the invention, associated with a shock wave generator of apparatus for detecting and destroying calculi;

FIG. 2 is a right side view of the imaging device of the invention associated with a shock wave generator of apparatus for detecting and destroying calculi, showing the different degrees of freedom to move in rotation of the device relative to an intangible center of rotation O that coincides with the second focus F2 of the wave generator;

FIG. 3 is a view analogous to the view of FIG. 2, showing an inclined position of the imaging device of the invention after the assembly formed by the imaging device and by the shock wave generator has moved;

FIG. 4 is a perspective view from below showing the echographic imaging device of the invention associated with a shock wave generator of apparatus for detecting and destroying calculi;

FIG. 5 is a diagrammatic view showing the structure of the moving arm for supporting the echographic probe of the imaging device of the invention, and its possibilities for adjusting the position and the length of the stroke along the longitudinal axis X-X′ of the arm; and

FIG. 6 is a view analogous to the view of FIG. 5, showing a second position for adjusting the length of the stroke of the moving arm of the echographic probe of the imaging device of the invention.

### MORE DETAILED DESCRIPTION

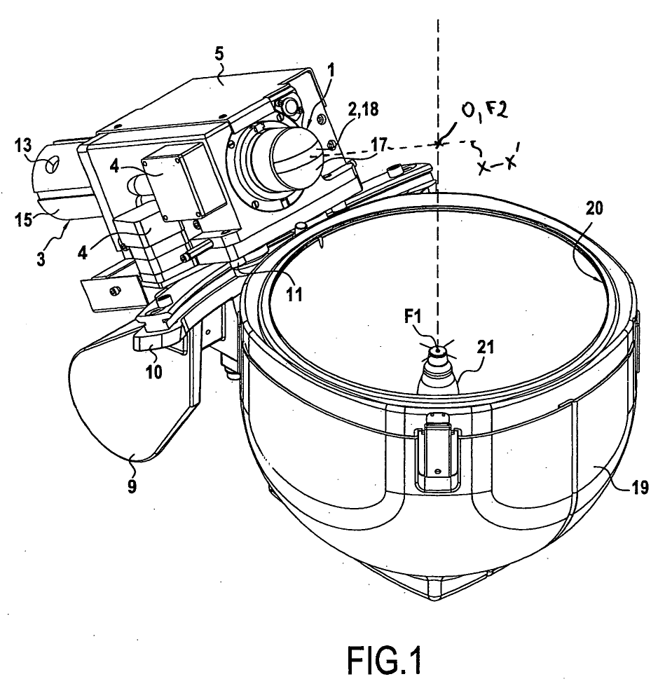

FIGS. 1 to 4 are various views of an echographic imaging device of the invention associated with a shock wave generator as commonly used in apparatus for detecting and destroying calculi in a human body or in an animal's body. Although the imaging device of the invention is not reserved or designed specifically for such a use in viewing and destroying calculi, it is particularly advantageous for such a use, and the following description is given with reference to said use.

In the preferred embodiment shown, the echographic imaging device of the invention advantageously comprises an echographic probe 1 incorporating an ultrasound transducer 2 that is, in conventional manner, connected electrically to a current generator (not shown) for generating ultrasound waves that, when the probe 1 is applied against the body of a patient, make it possible to obtain en echographic image of that plane of the body in which the ultrasound waves spread out.

In accordance with the invention, the echographic probe 1 is carried by a straight arm 3, of longitudinal axis X-X′, and mounted to move in translation therealong and in rotation thereabout via motor-driven displacement means 4. The moving arm 3 is mounted to slide on a frame 5 to which the motor-driven displacement means 4 for moving the arm 3 are also fastened.

The imaging device of the invention further comprises electrical control means for controlling the motor-driven displacement means 4, and force-monitoring means for measuring the application pressure with which the probe moved by the moving arm is applied against the surface of a patient's body.

The force-monitoring means may, in particular, be constituted by force sensors disposed on the arm 3, or in the echographic probe 1 itself, or indeed associated with the motor-driven displacement means for moving the arm 3. Such force sensors can, for example be constituted by strain gauges or indeed by sensors of the Force Sensing Resistor™ (FSR™) type.

In addition to the control means and to the force-monitoring means, the device of the invention further comprises servo-control means for servo-controlling the motor-driven displacement means 4 for moving the arm 3 that supports the echographic probe 1. In co-operation with the control means, with the force-monitoring means, and with the motor-driven displacement means 4 for moving the moving arm, said servo-control means enable the application pressure with which the echographic probe 1 is applied to be maintained constant independently of the movements of the patient's body and of the movements of the probe over said body.

In accordance with a preferred embodiment of the device of the invention, the motor-driven displacement means for moving the moving arm 3 comprise motors 4 of the servo-motor type such as the motors referenced Coolmuscle, from Myostat Motion (Canada). Said motors 4 advantageously have means for monitoring and for servo-controlling their power supply voltage and/or their power supply current relative to a setpoint value determined as a function of the application pressure to be procured for the echographic probe. In particular, said motors incorporate programmable microprocessors that can simultaneously perform the functions of force-monitoring and of servo-controlling the voltage and/or current of the motors in order to adjust and regulate the movements of the arm 3 and the application pressure with which the echographic probe 1 is applied against a patient's body.

Thus, by means of such programmable motors 4, the force-monitoring means and the servo-control means can be incorporated into the motor-driven displacement means 4 for moving the moving arm 3 carrying the probe 1.

In a variant, the force-monitoring means and the servo-control means can also be incorporated into the control means which, as is conventional in the field of medical instruments, comprise computer hardware and software means.

Said computer hardware and software means make it possible, inter alia, to set and to monitor the application pressure to be procured for applying the echographic probe 4 against a patient's body, in particular as a function of the morphology of said patient. They are also suitable for controlling the motor-driven displacement means 4 for moving the moving arm 3 supporting the probe 1 so that said probe is applied against the patient's body with the pressure suitable for obtaining good coupling between the probe and the patient's body and thus good echographic images, which images are also obtained and processed by computer hardware and software means that are well known in the field of echography techniques.

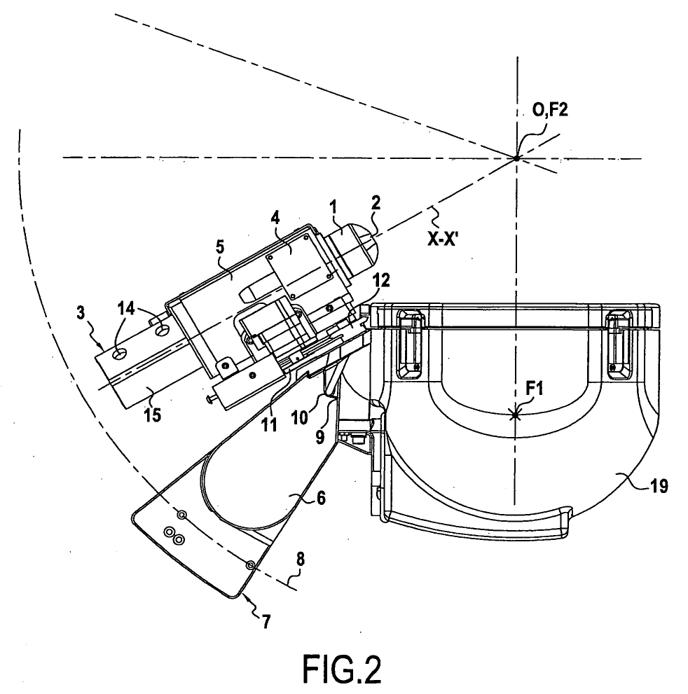

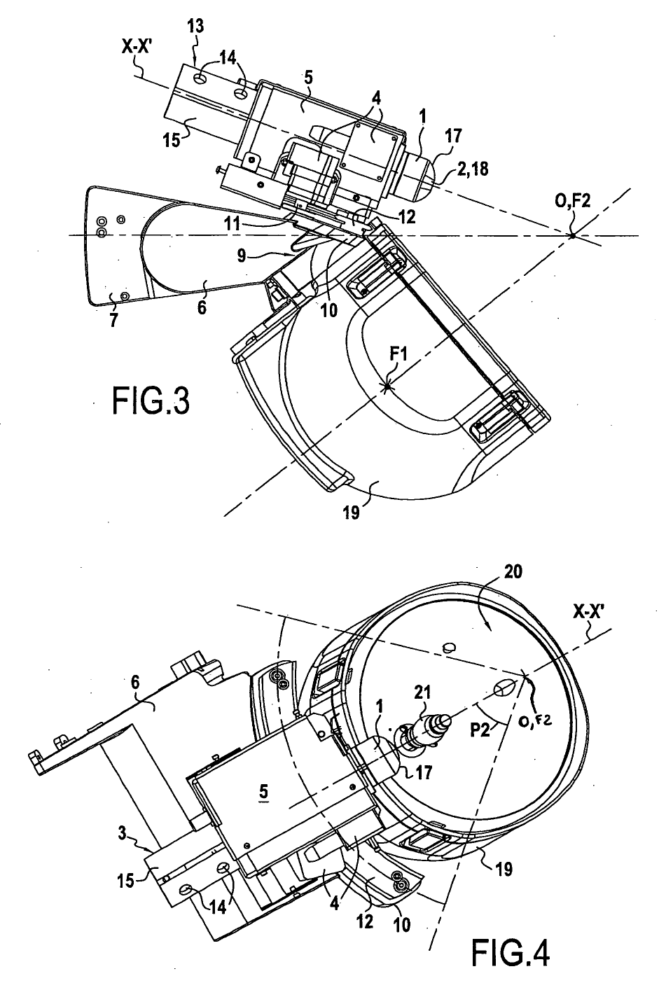

As appears in particular in FIGS. 2 and 3, the frame 5 on which the moving arm 3 carrying the probe 1 is mounted is itself mounted to move in rotation at least in a first plane P1 containing the axis X-X′ of the arm 3 and in a second plane P2 that that is substantially perpendicular to the plane P1, relative to the same intangible center of rotation O situated on the longitudinal axis X-X′ of the moving arm 3 carrying the probe 1.

This freedom to move in the perpendicular planes P1 and P2 about the same intangible center of rotation O is made possible, in the example shown, by the fact that the imaging device of the invention is provided with a support bracket 6 mounted to slide via a first end 7 provided with suitable fastening means for fastening it to a circular guide rail 8, represented in FIG. 2 by a circular arc drawn in axis lines, so as to guide the moving arm 3 and the echographic probe 1 in rotation in the plane P1 about the intangible center of rotation O.

Preferably, the extent to which the bracket can move in rotation in the plane P1 covers an angular sector of at least 50° relative to O.

In addition, as appears from FIG. 4, the bracket 6 also supports a plate 10 that is secured to its second end 9 and that supports a carriage 11 fastened to the frame 5 to which the moving arm and the echographic probe are fastened. As can be seen in FIGS. 1 and 3, in particular, the carriage 11 is mounted to slide on a circular guide rail 12 formed on or fastened to the plate 10 in such a manner as to guide the moving straight arm 3 and the probe 1 in rotation in the plane P2 perpendicular to P1 about the intangible center of rotation O.

In the plane P2, it is desirable for the carriage to make it possible to cover an angular sector of at least 30° relative to O.

In order to move along the guide rails 8 and 12, the bracket 6 and the carriage 11 are advantageously provided with motor-driven displacement means (not shown in the figures) that are connected to the electrical control means for controlling the device of the invention and that are suitable for co-operating with said circular guide rails 8, 12.

The motor-driven displacement means for moving the bracket 6 and the carriage 11 carrying the frame 5 advantageously make it possible to manipulate and move automatically the echographic probe 1 and the arm 3 that carries it and that moves it in rotation about the point O automatically relative to a patient's body, without the patient needing to move on the mattress or on the table on which said patient is recumbent during the examination and without the practitioner performing the examination needing to move the probe 1 in order to modify the aiming axis thereof.

Thus, the echographic imaging device of the invention enables the echographic probe to be moved in automated manner in three distinct movements in rotation and in one movement in translation along the axis X-X′, thereby enabling the probe to be positioned rapidly and accurately, with the application pressure that is necessary and sufficient to obtain satisfactory coupling and satisfactory images with good resolution, without discomfort for the patient and without any manual operation by the practitioner performing the examination on the patient. The value of each of the movements is measured in order to determine the three-dimensional position of the probe relative to any reference frame whose origin can be the point O.

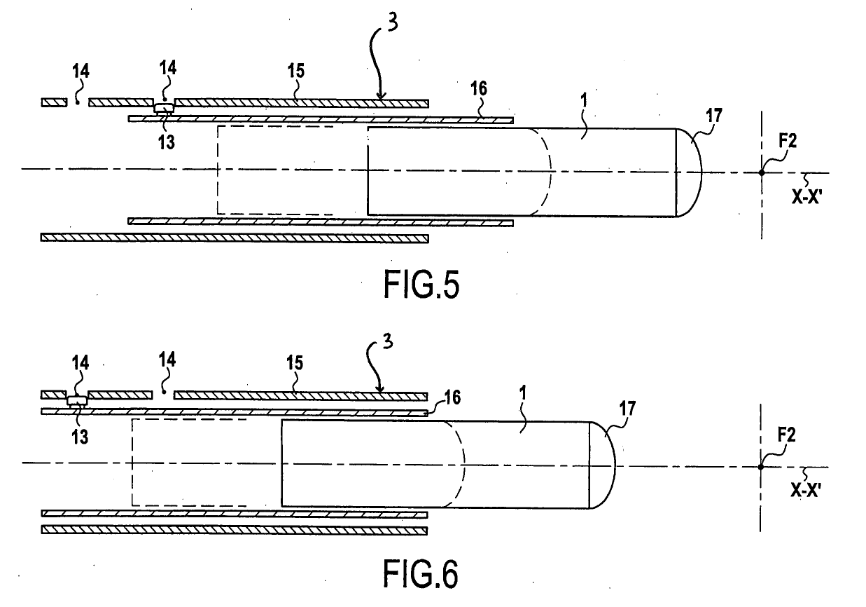

In accordance with another advantageous characteristic and as shown in FIGS. 5 and 6, the imaging device of the present invention is provided with adjustment means 13, 14 for adjusting the position of the moving arm 3 carrying the echographic probe 1 relative to the frame 5 and for adjusting the length of its stroke in translation along its longitudinal axis X-X′.

In particular, said adjustment means serve to make it possible to adapt the length of the arm 3 and of its stroke along the axis X-X′ so that it is possible to perform an echographic examination on any type of patient, child or adult, thin or obese.

For this purpose, in the preferred embodiment shown in FIGS. 5 and 6, the moving arm 3 is made up of at least two telescopic tube segments 15, 16 mounted to slide relative to each other, the tube segment 15 forming a sheath inside which it is possible to adjust the position of the second tube segment 16 that carries the echographic probe 1. In order to lock the two segments of tubes 15, 16 in translation relative to each other, and thus in order to make it possible to adjust the position of the arm 3 and the length of its stroke in translation, the adjustment means comprise at least one male locking means 13 and at least one female locking means 14, formed on respective ones of the telescopic tube segments 14, 16 of the moving arm. For example, as shown in FIGS. 5 and 6, the male locking means 13 can be constituted by a stud or push-button fastened to the tube segment 16 and suitable for co-operating with at least one, and preferably two female locking means 14 formed by orifices provided in the sheath 15 of the arm 3.

Thus, when the stud or button 13 on the segment 16 is pushed in, it is possible to cause the segment 16 of the arm 3 to slide inside the sheath 15 so as to increase or reduce the length of the arm 3 and so as to modify the “base” position of the echographic probe 1, and therefore so as to adjust the length of stroke in translation of the arm 3 along the axis X-X′. When the stud 13 on the segment 16 is in correspondence with one of the orifices 14 in the sheath 15, said stud then rises up into the orifice 14 under drive from a spring, thereby locking the two telescopic segments 15, 16 of the arm 3 relative to each other.

This mechanism of an arm having two segments 15, 16 makes it possible to obtain a mechanical offset chosen by the practitioner depending on the corpulence of the patient.

The above-descried motor-driven means 4 are the means making it possible to move the probe 1 in the sheath 16 so as to enable the application contact pressure with which the probe is applied against the patient to be controlled.

By means of these simple features, the imaging device of the invention advantageously makes it possible for practitioners to adjust the length of the arm 3 carrying the echographic probe to match the morphology of each patient so as to guarantee that the quality of the coupling between the probe and the patient's body is as good as possible.

Finally, in the preferred embodiment of the echographic imaging device of the invention that is shown herein, the echographic probe of the imaging device is also provided with a spherical or hemispherical applicator cup 17 so as to procure an application surface for the probe that is devoid of any unevenness or sharp edges. The cup 17 makes it possible to facilitate bringing the probe into contact with and sliding it over the patient while adjusting the position of the probe while the arm carrying it is moving in translation along its axis X-X′ and/or in rotation about said axis X-X′, and in rotation in the planes P1 and P2.

In particular, the cup 17 can clip onto the end of the probe. It can be disposable and thus designed for use once only, or else it can be washable and re-usable.

Advantageously, it has at least one zone 18 that is transparent to ultrasound, which zone is preferably disposed facing the ultrasound transducer 2 and of shape complementary to the shape thereof. This transparent zone 18 can, in particular, be formed by an opening of shape complementary to the shape of the echographic transducer of the probe 1.

In addition to its function of assisting sliding and coupling of the probe on patients' bodies, said cup 17 also constitutes a safety element that is made necessary by the automatic servo-controlling of the application pressure of the probe 1 that is made possible by the device of the invention and that is not under the control of practitioners.

The echographic imaging device of the invention as described above is essentially dedicated to medical uses. It is, in particular, designed especially to satisfy the needs of practitioners in extracorporeal lithotripsy treatments of renal, gall bladder, or salivary calculi, or of bone or cartilage structures.

Such treatments require, in particular, the use of echographic imaging means for identifying and tracking in real time the calculi and changes thereto in the patients' bodies during the treatment.

In this context, the present invention also proposes apparatus for detecting and destroying solid intracorporeal concretions that incorporates an echographic imaging device as described above and as shown in FIGS. 1 to 6.

The echographic imaging device of the invention makes it possible to view the calculus continuously and thus, in particular, to track in real time the concretions under treatment by guaranteeing continuously, by means of the moving arm 3 carrying the echographic probe 1 whose application pressure with which it is applied against the patient's body is monitored and servo-controlled automatically, that coupling is ideal between the echographic probe 1 and the patient's body, independently of any unwanted movements of the patient or of the probe.

In conventional manner, the apparatus of the invention for detecting and destroying intracorporeal concretions comprises a fixed structure supporting means for imaging intracorporeal concretions located in the body of a person or of an animal, and an acoustic shock wave (or pressure wave) generator shown in FIGS. 1 to 4 and designated by reference 19 in those figures. In conventional manner, the generator 19 is configured to emit acoustic shock waves that are directed towards a target focus F2 that it is desired to cause to coincide with the concretions, via the imaging means, so as to reach and destroy said concretions.

As mentioned above, the imaging means of the apparatus of the invention for detecting and destroying intracorporeal concretions comprise an echographic imaging device of the present invention. Additionally, preferably, and in conventional manner, said imaging means further comprise X-ray imaging means.

The association of X-ray imaging means with the echographic imaging device of the invention is particularly pertinent since certain calculi are X-ray transparent or indeed echo-transparent, and it is thus necessary, in order to identify them, to have the imaging means making such detection possible available. The X-ray imaging means also deliver images that are sometimes easier to interpret than the images obtained by echography. Often, it is advantageous for the information obtained by one type of imaging to be confirmed by the other type of imaging.

Preferably, and as shown in FIGS. 1 to 4, the acoustic shock wave generator 19 comprises an ellipsoidal reflector 20 in which an electrode 21 is positioned that makes it possible to generate an acoustic shock wave at the first focus F1 of the ellipsoidal reflector 20. By means of the particular geometrical shape of the reflector 20, the shock wave generated at F1 by the electrode 21 is reflected to the second focus F2 of the reflector 20, which focus F2 lies outside the generator, thus making it possible to aim at concretions situated in the body of a patient or of an animal by putting the concretions and the focus F2 of the generator into correspondence.

In accordance with a characteristic of the apparatus of the invention for detecting and destroying solid intracorporeal concretions, the echographic imaging device is mounted secured to the structure of the apparatus so that the intangible center of rotation O that lies on the longitudinal axis X-X′ of the echographic probe 1 and of its moving arm 3, coincides with the second focus F2 of the reflector of the shock wave generator.

In particular, as shown in FIGS. 1 to 4, it is particularly advantageous for the shock wave generator to be fastened to the second end 9 of the bracket 6 of the echographic imaging device in such a manner as to be movable in rotation simultaneously with the echographic probe 1 and with the moving arm 3 carrying it in the plane P1 relative to the second focus F2 of the shock wave generator that coincides with the intangible center of rotation O.

Thus, in order to track the concretions in the patient's body in real time, the apparatus of the invention makes it possible to move the echographic probe 1 and the generator 19 simultaneously in rotation about the intangible center of rotation O that coincides with the target focus F2 that itself coincides with a concretion to be destroyed inside the body of a patient. By means of the arm 3 of the echographic probe 1 being moved automatically, and by means of the application pressure with which said probe is applied to the body being controlled, also automatically, as made possible by the imaging device of the invention, it is thus possible to obtain continuously the best echographic images of the concretions under treatment and of their positions in the patient's body, while always having the generator 19 in an appropriate firing position.

By moving the generator 19 in rotation about the intangible point O, it is possible to seek the best approach route for the shock waves generated by it to the concretions to be destroyed, by making it possible to avoid tissue structures to be preserved or bone structures forming obstacles to ultrasound. The apparatus of the invention advantageously procures the possibility for the practitioner performing the examination to cause the target focus F2 and the shock wave focusing cone to appear on the echographic images obtained by using the echographic imaging device. Constraining the echographic probe 1 and the generator 19 to move together in rotation makes it possible to keep the same relative incidence of the focusing cone relative to the echographic imaging axis X-X′.

In order to make it easy for the target focus F2 of the generator to be caused to coincide with the concretions to be destroyed in the body of a person or of an animal, and above all in order to ensure that the patient under treatment is in as comfortable a position as possible during the treatment, the apparatus of the invention conventionally has a rest and positioning table (not shown) for receiving the body and for positioning it relative to the shock wave generator 19.

Advantageously, in accordance with the present invention, said table is equipped with displacement means adapted to move the body of the patient under treatment without the patient having to move, so as to position an intracorporeal concretion to be treated exactly in correspondence with the second focus F2 of the shock wave generator 19.

Said displacement means for moving the table of the apparatus can, in particular, comprise electric actuators controlled by manual or computerized electrical control means so as to move the table in three orthogonal directions X, Y, Z in space.

In particular, after the position of the concretion to be aimed at has been detected via the imaging means of the apparatus, be it via the echographic probe or via the X-ray imaging means, such actuators enable the table supporting the body of the patient under treatment to be moved automatically so as to position said patient relative to the generator such that a concretion to be destroyed is positioned exactly at the target focus F2 of the generator 19.

Naturally, while the table is being moved, the practitioner performing the treatment can observe the focus F2 being put into correspondence with the concretion to be destroyed in real time on monitoring screens showing the images obtained by the imaging means, and can, when necessary, use a manual remote control or indeed touch-sensitive control means on the screens to adjust manually the positioning of the table and of the generator 19 so that the firing window is as good as possible.

The apparatus of the invention for detecting and destroying solid intracorporeal concretions, which apparatus includes an echographic imaging device of the invention, thus makes it possible to optimize the quality of the echographic images obtained in extracorporeal lithotripsy treatment, and to optimize the comfort and the simplicity of implementation for the practitioner and of treatment for the patient.

The invention is not limited to the examples described and shown because various modifications can be made to it without going beyond its ambit.

1. An echographic imaging device comprising at least one echographic probe incorporating an ultrasound transducer connected electrically to a current generator for generating ultrasound waves, wherein said echographic imaging device further comprises a straight arm of longitudinal axis X-X′ and supporting the echographic probe, which arm is mounted to move in translation along its longitudinal axis X-X′ and in rotation thereabout via motor-driven displacement means, and is mounted on a frame that is mounted to move in rotation at least in a first plane P1 and in a second plane P2 that is substantially perpendicular to the plane P1 and about the same intangible center of rotation O situated on the longitudinal axis X-X′ of the moving arm carrying the probe, the intangible center of rotation O and the longitudinal axis X-X′ being contained in the planes P1 and P2, and wherein it further comprises electronic control means for electronically controlling the motor-driven displacement means, and force-monitoring means for measuring the application pressure with which the probe as moved by the moving arm is applied against a surface, in particular against a patient's body, and servo-control means for servo-controlling the displacement means for moving the arm so as to maintain said application pressure constant independently of the movements of the surface and of the movements of the probe relative to said surface.

2. A device according to claim 1, further comprising a support bracket mounted to slide via a first end provided with suitable fastening means on a circular guide rail so as to guide the echographic probe and the moving arm carrying it in rotation in the plane P1 about the intangible center of rotation O, said bracket supporting, secured to its second end, a plate that supports a carriage fastened to the frame to which the moving arm and the echographic probe are fastened, the carriage being mounted to slide on a circular guide rail formed on the plate so as to guide the moving straight arm and the probe in rotation in the plane P2 about the intangible center of rotation O.

3. A device according to claim 2, wherein the bracket and the carriage are provided with motor-driven displacement means connected to the electronic control means and suitable for co-operating with the circular guide rails of the bracket and of the carriage, respectively.

4. A device according to claim 1, wherein the force-monitoring means and the servo-control means are incorporated into the motor-driven displacement means for moving the moving arm carrying the probe and/or into the control means.

5. A device according to claim 1, further comprising adjustment means for adjusting the position of the moving arm carrying the echographic probe and the length of its stroke in translation along its longitudinal axis X-X′.

6. A device according to claim 5, wherein the moving arm comprises at least two telescopic tube segments mounted to slide relative to each other, and wherein the adjustment means for adjusting the position of the arm and the length of stroke in translation of the moving arm comprise at least one male locking means and at least one female locking means formed on respective ones of the telescopic tubes of the moving arm.

7. A device according to claim 1, wherein the displacement means for moving the moving arm in translation along its longitudinal axis X-X′ and in rotation thereabout comprise programmable motors having means for monitoring and servo-controlling their power supply voltage and/or their power supply current relative to a setpoint determined as a function of the application pressure to be procured for the echographic probe.

8. A device according to claim 1, wherein the control means comprise computer hardware and software means for adjusting and monitoring the application pressure to be procured for applying the echographic probe against a surface, and suitable for controlling the motor-driven displacement means for moving the moving arm supporting the probe.

9. A device according to claim 3, wherein the control means are suitable for controlling the displacement means for moving the bracket and the carriage so as to activate the movements in rotation of the frame in the planes P1 and P2 in rotation about the intangible center of rotation O.

10. A device according to claim 1, wherein the echographic probe is provided with a spherical or hemispherical applicator cup presenting at least one zone that is transparent to ultrasound, that is disposed facing the ultrasound transducer, and that is of shape complementary to the shape thereof so as to procure an application surface for the probe that is devoid of any unevenness or sharp edges.

11. Apparatus for detecting and destroying solid intracorporeal concretions such as renal, gall bladder, or salivary calculi, or bone or cartilage structures, said apparatus comprising a fixed structure supporting imaging means for imaging intracorporeal concretions located in the body of a person or of an animal, and an acoustic shock wave generator configured to reach and destroy said intracorporeal concretions located in the body of a person or of an animal, wherein the imaging means comprise an echographic imaging device according to claim 1.

12. Apparatus for detecting and destroying solid intracorporeal concretions such as renal, gall bladder, or salivary calculi, or bone or cartilage structures, said apparatus comprising a fixed structure supporting imaging means for imaging intracorporeal concretions located in the body of a person or of an animal, and an acoustic shock wave generator configured to reach and destroy said intracorporeal concretions located in the body of a person or of an animal; wherein the acoustic shock wave generator comprises an ellipsoidal reflector and an electrode positioned in the reflector in such a manner as to generate an acoustic shock wave at the first focus F1 of the reflector so that said wave is reflected to the second focus F2 of the reflector, and wherein it further comprises an echographic imaging device according to claim 2, secured to the structure of the apparatus so that the intangible center of rotation O coincides with the second focus F2 of the reflector of the shock wave generator.

13. Apparatus for detecting and destroying solid intracorporeal concretions according to claim 11, further comprising a rest and positioning table for receiving the body of a person or of an animal and for positioning it relative to the shock wave generator, said table being equipped with displacement means adapted for moving the body of a person or of an animal in such a manner as to position a said intracorporeal concretion exactly at the second focus F2 of the shock wave generator.

14. Apparatus for detecting and destroying solid intracorporeal concretions according to claim 11, wherein the imaging means further comprise X-ray imaging means.

15. Apparatus for detecting and destroying solid intracorporeal concretions such as renal, gall bladder, or salivary calculi, or bone or cartilage structures, said apparatus comprising a fixed structure supporting imaging means for imaging intracornoreal concretions located in the body of a person or of an animal, and an acoustic shock wave generator configured to reach and destroy said intracorporeal concretions located in the body of a person or of an animal, including an echographic imaging device according to claim 2, and wherein the shock wave generator is fastened to the second end of the bracket of the echographic imaging device so as to be movable in rotation simultaneously with the echographic probe and with the moving arm carrying it in the plane P1 relative to the second focus F2 of the shock wave generator, which focus coincides with the intangible center of rotation O.
